(19) United States
(12) Patent Application Publication
(10) Pub. No.: US 2014/0343407 A1
(43) Pub. Date: Nov. 20, 2014

(54) METHODS FOR THE ASSISTED MANIPULATION OF AN INSTRUMENT, AND ASSOCIATED ASSISTIVE ASSEMBLY

(71) Applicant: GENERAL ELECTRIC COMPANY, Schenectady, NY (US)

(72) Inventors: Serge Muller, Buc (FR); Laurence Vancamberg, Buc (FR); Razvan Iordache, Buc (FR); Guillame Morel, Buc (FR); Anis Sahbani, Buc (FR)

(73) Assignee: GENERAL ELECTRIC COMPANY, Schenectady, NY (US)

(21) Appl. No.: 14/359,662

(22) PCT Filed: Nov. 21, 2012
(86) PCT No.: PCT/US2012/066330

§ 371 Date: May 21, 2014

(30) Foreign Application Priority Data
Nov. 21, 2011 (FR) 1160609

(51) Int. Cl. A61B 19/00; A61B 5/06

(52) U.S. Cl. CPC A61B 19/20; A61B 5/061
USPC 600/424

(57) ABSTRACT

A method for the assisted manipulation of an instrument in an assistive assembly for instrument manipulation, comprising: a medical imaging system comprising a support to immobilize at least one part of a patient intended to be imaged or already imaged by said system, an assistive device for assisted manipulation of an instrument intended to be used in said patient, said device comprising: a mobile mechanical structure operable by an operator, on which at least one instrument can be fixed, said method being characterized in that it comprises the step to calibrate the position of the assistive device relative to the position of the imaging system, thereby allowing the position of the patient part to be known within a reference frame related to the assistive device, to assist the operator in manipulating the instrument in the patient.

### GENERAL TECHNICAL FIELD

Embodiments of the present invention relate to a device and assembly for the assisted manipulation of an instrument intended to be used in a patient.

### STATE OF THE ART

Surgical procedures, in particular minimally invasive procedures such as biopsies, require the manipulation of instruments by the physician. These instruments must be moved and positioned in precise regions for proper conducting of the procedure. These may be needles for example which are to be positioned at a precise site in the patient to perform a biopsy.

The manipulation of these instruments is a delicate and complex operation.

For this purpose, it is known to provide devices for assisted instrument manipulation.

For example, in surgery, it is known to use jointed arms which pre-position the end of the arm on which an instrument is articulated. Once the arm is thus pre-positioned, the user can cause the instrument to perform a particular movement in translation or rotation whilst the arm remains held in its position.

The devices may also be simpler devices, such as mechanical guides.

The devices for assisted instrument manipulation known to date have numerous shortcomings.

To define the trajectory to be imparted to an instrument, some devices require the identification of anatomical markers in the patient regions to be investigated. The identification of these anatomical markers allows a positional reference frame of the device to be calibrated relative to the patient's body. This positioning is necessary in order to be able to determine the trajectories to be given to the instrument.

At times, the calibration of the positional reference frame of the device relative to the patient's body, which is also called registration, is performed by using a three dimensional model (3D) of the organ. This is notably the case with known navigation and assisted manipulation systems in orthopaedic surgery. In this case, a 3D model of the target organ or organs can be constructed pre-operatively, and the trajectories can be determined in relation to these models before surgery during a so-called surgical planning phase. At the time of the surgical procedure, an instrument e.g. optical or magnetic is used to align the preoperative 3D model with the patient and to determine its location in relation to the device.

In all cases, registration is complex and sometimes imprecise. In addition it is not always applicable. Finally, it has to be performed again in the event of patient or organ movement.

Also, it is often necessary to have a mechanical guide allowing the instrument to be guided over a precise trajectory. These mechanical guides are used, for example, for the positioning of needles.

This solution has the disadvantage that it requires an additional part. Also, it is necessary to disinfect the part for each patient. Finally, this part is not adapted for all instruments and only allows a limited set of trajectories to be defined.

Embodiments of the invention propose improving on the devices for assisted instrument manipulation known to date.

### DISCLOSURE OF THE INVENTION

Embodiments of the invention propose overcoming the above-mentioned disadvantages.

In one embodiment, a method is described for the assisted manipulation of an instrument, in an assembly for assisted instrument manipulation, comprising a medical imaging system including a support for immobilizing at least one part of a patient to be imaged or already imaged by said system, a device for the assisted manipulation of an instrument intended to be used in said patient, said device comprising a mobile mechanical structure operable by an operator, on which at least one instrument can be fixed, said method being characterized in that it comprises the step of calibrating the position of the assistive device relative to the position of the imaging system, thereby allowing the position of the patient part to be known within a reference frame related to the assistive device, to assist the operator when manipulating the instrument in the patient.

In one embodiment, the step consists of providing the operator with information characterizing the position of the assistive device and/or the instrument relative to the patient region or to a target of this patient region.

In one embodiment, the assistive assembly further comprises motorizations actuating the mechanical structure over one or more degrees of freedom, and a processor capable of driving the motorizations to facilitate the meeting of at least one kinematic constraint on the instrument, the meeting of the constraint being achieved by cooperation between manipulations by the operator and actions by the motorizations in response to these manipulations, said calibration enabling the processor to transform the kinematic constraint on the instrument defined in the reference frame of the imaging system into the reference frame of the device so as to drive the motorizations using said transformed constraint.

According to one embodiment, the device and the imaging system are combined and the calibration is pre-programmed.

In one embodiment, the calibration is determined by the operator prior to procedure in the patient, and comprises the steps of positioning the device at points of the imaging system, the position of these points in the reference frame of the imaging system being transmitted to a system calibrating the position of the assistive device relative to the position of the imaging system, and inferring therefrom the calibrated position of the device relative to the imaging system, via processing by the calibration system.

In one embodiment, the processor is configured to drive the motorizations so as to compel the instrument to position itself within a guide, corresponding to a geometric spatial region.

In one embodiment, the guide is defined dynamically in relation to kinematic parameters of the instrument and/or to the manipulations of the instrument performed by the operator.

In one embodiment, the guide is defined dynamically in relation to a deformation model of the patient region in which the instrument has been inserted.

In one embodiment, the guide is re-computed in relation to data derived from images of the patient, taken by the medical imaging system.

In one embodiment, the guide corresponds to a geometric region enabling the instrument, when being moved by the operator, to avoid physical objects or predetermined regions of the patient.

In one embodiment, the kinematic constraint on the instrument is defined by an operator from images provided by the medical imaging system, via an interface of the assistive assembly.

An assistive assembly is also described to assist in manipulating an instrument, comprising a medical imaging system including a support for immobilizing at least part of a patient intended to be imaged or already imaged by said system, an assistive device for the assisted manipulation of an instrument intended to be used in said patient, said device comprising a mobile mechanical structure which can be operated by an operator, on which at least one instrument can be fixed, said assembly being characterized in that it comprises at least one calibration system to calibrate the position of the assistive device relative to the position of the imaging system, thereby allowing the position of the patient part to be known within a reference frame related to the assistive device to assist the operator in manipulating the instrument in the patient, said assembly being capable of performing steps of the previously described method.

In one embodiment, the assembly further comprises motorizations actuating the mechanical structure over one or more degrees of freedom, and a processor capable of driving the motorizations to facilitate the meeting of at least one kinematic constraint on the instrument, the meeting of the constraint being achieved by cooperation between manipulations by the operator and actions by the motorizations in response to these manipulations, said assembly being characterized in that it is capable of performing steps of the previously described method.

A computer program product is also described, which can be loaded into a memory of a processor of an assistive device of an assistive assembly, said programme being capable of controlling the processor to perform the steps of the previously described method.

One advantage of an embodiment of the invention is to propose a device for the assisted manipulation of an instrument providing the operator with freedom of movement.

Another advantage of an embodiment of the invention is to allow real-time visualization of the operator's manipulations, which improves the precision of the instrument trajectories and patient safety.

A further advantage of an embodiment of the invention is to offer flexible assistance for the guiding of an instrument, capable of being adapted to the patient and to the presence of physical objects in the environment of the procedure. An embodiment of the invention allows guiding over multiple trajectories that are adjustable in relation to various parameters.

A further advantage of an embodiment of the invention is that the positioning of the device can be determined in simple, efficient manner.

A further advantage of an embodiment of the invention is that it is possible to calculate a trajectory (or constraints) and to cause the instrument to follow the trajectory. The trajectory is, in an embodiment, chosen for example via a planning algorithm, and allows minimization of the regions of the patient's body to be opened for insertion and movement of the instrument, which improves patient comfort and reduces bleeding.

A further advantage of an embodiment of the invention is the discontinued need for guiding parts and the maintenance thereof.

Finally, another advantage of an embodiment of the invention is to allow the aligning of the instrument with a target whilst providing the operator with freedom of movement.

### PRESENTATION OF THE FIGURES

Other characteristics, objectives and advantages embodiments of the invention will become apparent from the following description which is purely illustrative and non-limiting, and is to be read with reference to the appended drawings in which:

FIG. 1 is a schematic illustration of an assistive assembly according to an embodiment of the invention;

FIG. 2 is a schematic illustration of steps of a method according to an embodiment of the invention;

FIG. 3 is a schematic illustration of steps of a method according to an embodiment of the invention;

FIG. 4 is a schematic illustration of a pointing constraint on an instrument to point towards a target;

FIG. 5 is a schematic illustration of trajectories of an instrument;

FIG. 6 is a schematic illustration of a guide varying in relation to the depth of insertion of the instrument;

FIG. 7 are a schematic illustration of a constraint allowing a point of insertion for the instrument to be maintained.

### DETAILED DESCRIPTION

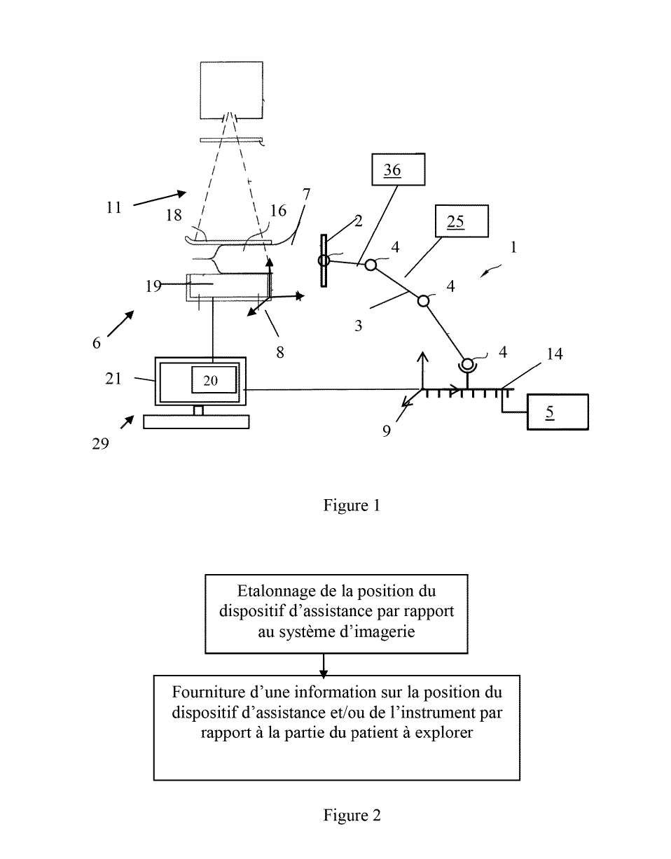

FIG. 1 schematically illustrates an assistive assembly 6 for manipulation of an instrument.

The assembly 6 comprises a medical imaging system 11 allowing images to be taken of a patient 7.

This may be a mammography unit for example in which a patient is positioned for the taking of mammography images.

The medical imaging system 11 comprises a support 18 for immobilizing at least one part 16 of a patient 7 to be imaged or already imaged by said system 11.

This support 18 is used to maintain immobile the part 16 of the patient 7 to be imaged, relative to the medical imaging system 11.

In FIG. 1, a mammography unit 11 is shown in which the breast 16 of the patient 7 is positioned immobile between a compression paddle 18 and a detector 19. In particular, the patient's breast 16 is compressed between the compression paddle 18 and the detector 19.

The conventional parts of the mammography unit are not described (X-ray source, etc.), and are known to the person skilled in the art.

The operator may for example perform a biopsy on the compressed breast of the patient 7.

The assembly 6 further comprises a device 1 for the assisted manipulation of an instrument 2.

The device 1 and the instrument 2 are configured to be used in a patient 7 who can be positioned at an imaging system 11.

The instrument 2 particularly includes any medical device which a physician may use to perform an examination, for example to sample tissue from an organ (biopsy) or to carry out a surgical procedure (needle, probe, etc.).

The device 1 comprises a mobile mechanical structure 3 on which at least one instrument 2 can be fixed. The mechanical structure 3 can be operated by an operator.

It may be a jointed arm for example.

In an embodiment, the mechanical structure 3 is itself articulated on a support 14. At its end opposite the support 14, the mechanical structure 3 carries the instrument 2.

In one embodiment, the support 14 is the medical imaging system 11 itself.

A said mechanical structure 3 is a poly-jointed system for example, such as the PHANToM Omni robot articulated arm distributed by SensAble Technologies, Inc., Woburn, Mass.

The mechanical structure 3 imparts the instrument 2 with a certain number of degrees of freedom. In FIG. 1, there are six degrees of freedom. There may be a different number depending upon applications, e.g. five degrees of freedom.

In some cases, the device 1 comprises a position sensor allowing determination of the position of the instrument 2 during a procedure. When applicable, the device 1 may comprise a velocity and optionally an acceleration sensor.

In one embodiment, a method for the assisted manipulation of an instrument, in the assistive assembly 6 for instrument manipulation, comprises the step of calibrating the position of the assistive device 1 relative to the position of the imaging system 11, thereby allowing determination of the position of the patient part 16 within a reference frame related to the assistive device 1, to assist the operator in manipulating the instrument in this part of the patient (cf. FIG. 2).

In one embodiment, this calibration is conducted once and for all, before each operative session in a patient, without the need to repeat this calibration in the course of a procedure. This provides information on the position of the assistive device 1 relative to the imaging system 11, and hence relative to the patient part 16 since this part 16 is immobile relative to the imaging system.

In one embodiment, this calibration can be pre-programmed (in a memory or a processor of the assembly), once and for all. This is particularly advantageous when the device and the imaging system are combined i.e. the device 1 and the imaging system 11 are mechanically connected. This may be the case for example when the device and the imaging system are sold together. In an embodiment, during the manufacture of the assembly, the device and the imaging system are calibrated together at the factory.

In general, the assistive assembly may comprise at least one calibration system 25 to calibrate the position of the assistive device 1 relative to the position of the imaging system 11, thereby allowing determination of the position of the patient part within a reference frame related to the assistive device 1, to assist the operator in manipulating the instrument in the patient.

The calibration system 25 optionally comprises any processor or processing unit needed for carrying out the described tasks. This system 25 may be fully integrated in the device 1, or it may be partly present in the device 1 and in the imaging system.

In one embodiment, before surgical procedure in a patient, an operator positions the device 1 at a plurality of points of the medical imaging system 11. The position of these points within the reference frame of the imaging system 11 is transmitted to the calibration system 25. This transmission is performed by the operator for example, or is performed automatically if the position of these points within the reference frame of the imaging system 11 is known and pre-recorded (in a memory of the processing assembly).

The calibration system 25 performs processing which allows deducing of the link between a reference frame of the imaging system 11 and a reference frame of the device 1, starting from known points of the medical imaging system 11, and using processing of the type used for a change in reference frame.

The known points are points of the compression paddle 18 for example, or points located on the detector 19. It is possible to use three points for example.

This calibration can even be determined without the need for the presence of the patient at the imaging system. The position of the patient with respect to the imaging system is known in advance on account of the presence of the immobilizing support.

The position of the device 1 is therefore calibrated relative to the imaging system 11 and the patient.

In general, the calibration system 25 may comprise any locating system, or any topographical recognition system allowing recognition of the characteristic points of the imaging system.

This may be, but is not limited thereto, a locating system using an optical field for example, an ultrasound locating system, or a mechanical positioning system.

One example of embodiment includes a radiofrequency receiver associated with one or more coils. The coils are arranged in the imaging system for example, and the receiver is arranged in the device 1, which allows locating of the position of the coils and hence determination of the relationship between the position of the imaging system and the position of the device 1. Once the calibration is completed, part of the device 1 remains fixed during the procedure in the patient e.g. the support of the device, to maintain the relationship between the reference frame of the device and that of the imaging system.

In another example of embodiment, the calibration system 25 comprises a camera fixed on the imaging system, which recognizes particular points of the assistive device 1, or conversely.

The calibration of the position of the device 1 is performed by calibration relative to the position of the imaging system 11 itself, and not relative to the image or the organ of the patient to be imaged, which is most advantageous and avoids the complex, scarcely precise methods of the prior art.

The calibration of the position of the assistive device 1 relative to the position of the imaging system 11 has numerous advantages for the operator. In one embodiment, the assistive method comprises the step of providing the operator with information characterizing the position of the assistive device and/or the instrument relative to the patient region to be investigated and positioned at the imaging system, or relative to a target in this patient region.

In one embodiment, the assistive assembly comprises a position informing system 36 allowing determination of the position of the instrument and allowing the operator to be informed on the position of the instrument and/or informing the operator on the difference between the current position of the instrument and the target to be reached.

This difference may be expressed in various forms: distance, angle, time, etc.

Knowing the position of the patient region within the reference frame of the device via the above-mentioned calibration, or of a target in this patient region, the position informing system 36 can provide the operator with feedback indicating the difference between the current position and the target to be reached.

This information-providing step can be conducted in various manners. In one embodiment, the position informing system 36 comprises a display screen providing visual information on the position of the assistive device or of the instrument relative to a region or target point in a patient. The information may also be a sound signal or any other suitable signal.

The position-informing system 36 may for example comprise an ultrasound position sensor, and/or an optical position sensor, and/or an electromagnetic position sensor, and/or a position sensor using laser, in order to know the position of the instrument.

The position-informing system 36 compares the position of the instrument measured by the position sensor with the position of the target to be reached, and then gives feedback information on the difference between these positions. The position of the target can be pre-programmed or determined using a method for trajectory calculation as explained below. The position-informing system 36 comprises any processor or processing unit needed to carry out the aforementioned tasks.

In one embodiment, the device 1 further comprises motorizations 4 actuating the mechanical structure 3 over one or more degrees of freedom. In FIG. 1 (nonlimiting for the invention) the device 1 comprises three motorizations.

The assistive device 1 further comprises a processor 5 capable of driving the motorizations to facilitate the observance of a kinematic constraint on the instrument 2.

The kinematics notably includes one or more of the following parameters: position, velocity, trajectory, direction, orientation of the instrument 2.

The term processor is to be construed in its broad meaning, and relates to any processing unit capable of emitting instructions and of receiving information to control the movements of the mechanical structure 3.

It may be a microcomputer for example associated with one or more control programs. It may optionally comprise a storage unit (memory).

The processor 5 is either integrated directly in the structure of the device 1, or it is external and communicates with the motorizations via wire or wireless communication means.

The processor 5 is capable of driving the motorizations 4 to facilitate the meeting of a kinematic constraint on the instrument 2, the observance of the constraint being achieved by cooperation between: manipulations by the operator, and actions by the motorizations 4 in response to these manipulations.

The most often, the cooperation between operator manipulations and actions by the motorizations is simultaneous.

The actions of the motorizations 4 tend to cause heed of the kinematic constraint, despite manipulations by the operator tending not to pay heed to this kinematic constraint.

It is therefore a co-manipulated device, both by the motorizations and by the operator's manipulations.

The kinematic constraint of the instrument 2 is met by cooperation between the operator's manipulations and the actions by the motorizations of the device.

The operator manipulates the instrument 2 during a procedure, and if such manipulation tends not to observe the kinematic constraint (e.g. it moves outside a predetermined geometric region) the motorizations prevent or tend to prevent the operator's movement which fails to pay heed to the constraint.

In an embodiment, but not limited thereto, the mechanical structure 3 applies a contrary force to the operator's movement.

The motorizations 4 may induce other actions on the mechanical structure, such as vibration, indicating that the operator's manipulations do not observe the kinematic constraint.

In an embodiment, the motorizations 4, under the control of the processor 5, are configured to generate blocking whose rigidity is adjustable so that the kinematic constraint can be met in cooperation with the operator's manipulations.

In another embodiment, a sound signal is also emitted by the device 1 to indicate to the operator that the kinematic constraint on the instrument 2 is not being observed. For this purpose, the device 1 comprises a speaker.

The operator is therefore provided with freedom for manipulating the instrument 2 and the device 1, whilst having the assurance that the device 1 will prevent failure to heed to the kinematic constraint on the instrument. The processor 5, associated with the motorizations 4, therefore provides a control loop for manipulation by the operator, of feedback loop type.

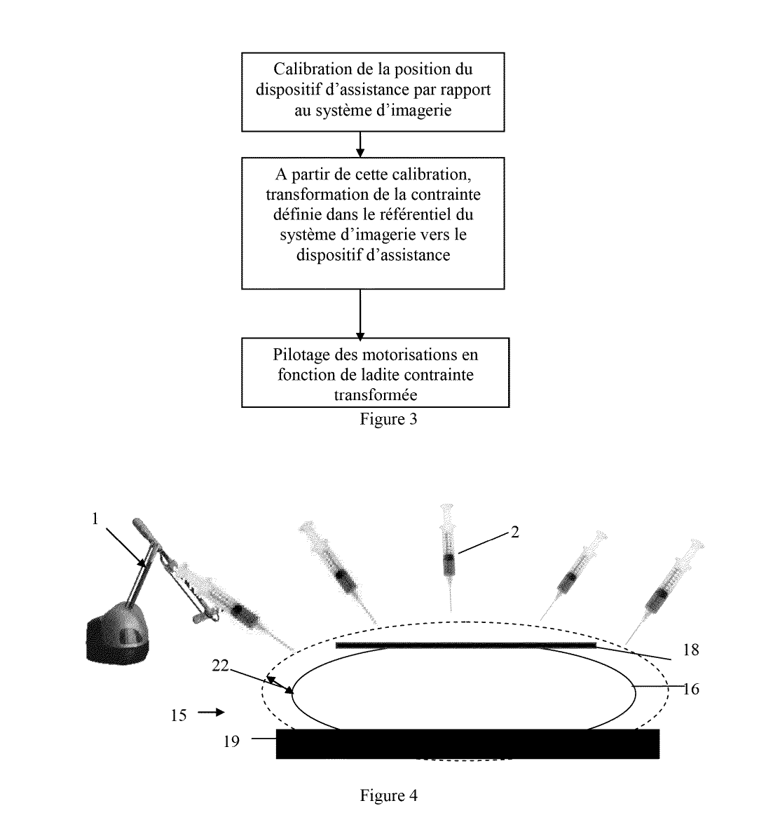

According to one embodiment, a position calibration method is described (cf. FIG. 3), comprising the step of calibrating the position of the assistive device 1 relative to the position of the imaging system 11, said calibration enabling the processor 5 to transform the kinematic constraint on the instrument defined in the reference frame of the imaging system into the reference frame of the device. The processor 5 is capable of driving the motorizations in relation to said transformed constraint.

As will have been understood, the driving of the device 1 must be made within the own reference frame 9 of said device 1. Therefore, the device 1 necessitates the expression of kinematic set points, such as trajectories, or positions, within its own reference frame.

Yet, the imaging system 11 provides an image of the region 16 of the patient 7 to be investigated which is defined within the reference frame of the imaging system, insofar as the region 16 of the patient 7 to be investigated by the instrument 2 and to be imaged is immobile relative to the imaging system 11.

The kinematic constraints on the instrument 2 are therefore determined by the user or by the assistive assembly 6 within the reference frame of the imaging system 11. For example, as described below, the assistive assembly 6 is able to determine trajectories to be avoided by identifying, in the images taken by the imaging system 11, those patient regions which must not be touched by the instrument 2 (vessels, organs, etc.). Initially, these trajectories are therefore defined within the reference frame of the imaging system 11.

The calibration of the position of the device 1 is performed here by calibration relative to the position of the imaging system 11 itself, and not relative to the image or to the organ of the patient to be imaged, which is very advantageous, and avoids the complex and scarcely precise methods of the prior art.

The fact that the processor 5 is capable of transforming a kinematic constraint of the instrument 2 expressed in the reference frame 8 of the imaging system 11 into a constraint expressed in the reference frame 9 of the device 1, allows the constraints determined by the assistive assembly or by the user of the assistive device to be applied within the device's own reference frame 8.

For example, if a trajectory towards a target in the patient's body has been determined for the instrument, said trajectory allowing the avoiding of blood vessels, this trajectory is transformed towards the reference frame of the device 1 by means of the aforementioned calibration, which enables the processor 5 to control the motorizations 4 so as to facilitate heed of this trajectory.

The calibration, making it possible to change over from the reference frame of the imaging system to the reference frame of the device, can be obtained via the aforementioned calibration system or via all the previously described embodiments and will not be further described.

One embodiment concerns a computer program product capable of driving the processor to perform calibration steps of the device relative to the imaging system 11, and to perform the previously described steps of transforming constraints defined in the reference frame of the imaging system into the reference frame of the device itself.

The assistive assembly 6 has numerous advantages. In one embodiment, the medical imaging system 11 allows visualization of the manipulation of the device 1 and/or of the instrument 2 by the operator. This is notably the case when the imaging system is a real-time imaging system. The real-time visualization of operator manipulations improves the precision of the trajectories chosen by the user, and patient safety.

Alternatively, it is possible to visualize the images taken by the system 11 and registered with respect to the device, on another monitor. Knowing the position of the instrument in real time, it is possible to follow the manipulation of the instrument in the volume of images previously acquired by the system, for example on the other monitoring screen. The operator is therefore able to visualize the navigation of the instrument within the volume of previously acquired images.

The assembly 6 may comprise a screen 21 which may be a dedicated visualization screen or the screen of the medical imaging system 1. Optionally, the visualization screen 21 allows display the images taken by the instrument 2 if the instrument 2 is a probe or a camera, the instrument being independent of the imaging system 11.

The operator is therefore able to follow its own manipulations of the instrument 2 in real time and to adapt the kinematic constraints it desires to impart to the instrument in relation to the images taken by the imaging system 11.

The unique combining of a co-manipulated device with an imaging system is therefore very advantageous.

If the imaging system 11 uses radiation (e.g. X-rays), the assembly 6 enables the operator to manipulate the instrument 2 via the device 1 whilst avoiding operator exposure to radiation in some embodiments.

For example, if the instrument is manipulated in “navigation” mode, i.e. the position of the instrument is visualized in the volume of already acquired images, it is not necessary to emit radiation at the same time as manipulation by the operator, which avoids exposure of the operator to radiation.

In an embodiment, the assembly comprises a selection interface 20 enabling the operator to select the kinematic constraint on the instrument 2 from the images taken by the imaging system 11. This may relate for example to the defining of a target or of a region to be reached in the patient. The operator may for example, in an image taken by the imaging system 11, directly select the target or trajectory it is desired to define for the instrument 2, even in real time.

In general, the assembly 6 may comprise a processing unit 29 of microcomputer type enabling a user to select guides, described below, for the instrument 2, and to control all the acquisition parameters of the imaging system 11 and the kinematic constraints placed on the instrument 2. In this case, the monitor screen 21 and the interface 20 belong to this processing unit 29. The processing unit is capable of communicating with the imaging system and the device.

According to one embodiment, which may or may not be complementary to the preceding embodiments, the processor 5 is configured to drive the motorizations 4 so as to compel the instrument 2 to position itself within a guide, corresponding to a geometric region in space.

These guides are determined for example by the processing unit 29 of the assembly 6, from the images taken by the imaging system 11.

As previously mentioned, the guide is defined within the reference frame of the imaging system, and is transformed by the processor into the reference frame of the device, by means of the prior position calibration of the device relative to the imaging system.

This may be any type of geometric region: volume, straight line, plane, etc.

It is therefore a virtual guide (as opposed to the mechanical guides of the prior art).

In an embodiment, the guide is defined dynamically i.e. it varies in relation to parameters. This therefore allows guiding over multiple trajectories which are adjustable in relation to various parameters. In one embodiment, the guide is defined dynamically in relation to: kinematic parameters (position, velocity, etc.) of the instrument 2, and/or manipulations of the instrument 2 performed by the operator.

For example, the guide may therefore vary in relation to the distance between the instrument 2 and a target to be reached. The closer the target to be reached, the more the geometric region in which the instrument is allowed to move is restricted.

In an embodiment, the guide is defined dynamically in relation to a deformation model of the patient region in which the instrument has been inserted.

The physical conditions for insertion of the instrument are dependent upon the chosen region (soft tissue, density, type of organ, etc.), and the insertion kinematic parameters. By having a deformation model of the region it is possible to fine-tune the kinematic constraints to be placed on the instrument, and hence the commands emitted by the processor 5 towards the motorizations 4.

In an embodiment, the guide is re-computed by the processing unit 29 in relation to the information derived from images of the patient taken by the imaging system 11.

It is indeed possible that the region of the patient in which the instrument has been inserted may undergo change (movement of the target, onset of new obstacles, etc.). In this case, the guide is re-computed by the assembly 6 taking this data into account.

According to one particular characteristic of the device 1, it is configured to hold the instrument 2 in position when the operator releases the instrument 2 or no longer exerts any force thereupon. This prevents falling of the instrument 2 during the surgical procedure.

In one so-called free mode, the device 1 does not apply any force on the instrument 2.

In general, the device 1 allows the providing of increased safety during procedures, whilst offering efficacy and comfort equivalent to mechanical guides of the prior art. The number of degrees of freedom offered to the operator is also increased. The assisted guiding of the instrument is flexible and is adapted to the patient and to the physical environment of the procedure (imaging system, connections, etc.).

### 1st Example of Embodiment

In this example of embodiment, the kinematic constraint on the instrument 2 is a guide compelling the instrument 2 to point towards a target, despite the manipulations of the operator (cf. FIG. 4). This allows alignment of the instrument with a target, whilst providing the operator with freedom of movement.

The target may be defined for example by the user, via the processing unit 29 of the assembly 6.

This embodiment is particularly useful to allow the operator to choose the point of insertion in the organ of a patient, and therefore the trajectory to be chosen to reach the target.

Therefore, the operator is able to move the instrument 2 around the patient's organ whilst the processor 5 actuates the motorizations 4 to urge the instrument to point towards the target. The motorizations 4 may in particular induce a force tending to deflect the instrument systematically towards the target. The target can be considered to be a virtual centre of rotation of the instrument.

By means of the controlling by the processor 5 over the kinematics of the instrument 2, the operator is able to move the instrument 2 within the entire space whilst being guided to point towards the target. It is therefore not the mechanical arrangement of the robot which allows directing towards the target but the control command sent by the processor 5 to the motorizations 4, which adapts in real time to the manipulations made by the operator. Therefore the operator can move the instrument in the entire space whilst paying heed to the desired kinematic constraint.

In an embodiment, the guide allows ensuring of the kinematic constraint compelling the instrument 2 to be positioned at a safe distance 22 from the organ or target. This prevents the involuntary touching of the organ or target with the instrument 2.

In addition, the operator has the assurance that the chosen point of insertion will be targeted, which is not the case when manipulations are solely manual. FIG. 1 illustrates the case of a patient positioned at a mammography unit 11 in which the patient's breast 16 is positioned immobile between a compression paddle 18 and a detector 19. The instrument 2 illustrated in FIG. 4 is a needle (case of a biopsy of the breast).

The assembly 6 comprising the device 1 and the imaging system 11 enables the operator to visualize the different possible entry points into the organ, and in particular to avoid regions of the organ incompatible with such insertion (vessels, region that is too narrow, beauty spots, etc.).

The association of the medical imaging system 11 with the device 1 is therefore most advantageous.

In an embodiment, the assistive assembly 6 allows the display (on screen 21) of each point of insertion for the optimal trajectories leading to the target. Therefore, when the operator moves the instrument towards a point of insertion the assembly 6 induces the display in the image, taken by the imaging system, of the optimal trajectories leading to the target.

In addition, the operator is able to lock the point of insertion in the device and the chosen trajectory. The recording of this trajectory will, at the time of insertion of the instrument, allow the applying of a kinematic constraint on the instrument allowing heed of insertion at this point of insertion and of the aforementioned trajectory.

As it will have been ascertained, this overcomes the need for mechanical guiding devices with poor precision and systems not allowing visualization of the image of the target to be reached and/or of the trajectories leading to this target.

### 2^{nd} Example of Embodiment

In this example of embodiment, the kinematic constraint is a guide corresponding to a trajectory to be imposed upon the instrument. The guide therefore allows this trajectory to be observed by the instrument.

This is used for example to conduct a biopsy or anaesthesia. The trajectory may be a straight line for example for a biopsy. In an embodiment, the trajectory is a trajectory recorded by the device 1, corresponding to a trajectory previously followed by the instrument. This may be useful for example for anaesthesia followed by a biopsy. It is desired in this case that the trajectory followed for the biopsy should be identical to the trajectory followed for anaesthesia.

Alternatively, it may be a planned trajectory, for example to avoid physical obstacles or predetermined regions of the patient. The guide within which the instrument must be confined is therefore defined to exclude these physical obstacles and these predetermined regions of the patient.

This planned trajectory can be determined by the processing unit of the assembly, from the images taken by the imaging system.

Planning algorithms are known in the state of the art, such as the document “Multicriteria trajectory planning for hepatic radiofrequency ablation” by Baegert et al., Medical Image Computing and Computer-Assisted Intervention—MICCAI 2007, or the document “Needle Insertion Parameter Optimization for Brachytherapy” by Dehghan et al., IEEE Transactions on Robotics, Col. 25, No. 2, April 2009, or patent application FR1001897 by the applicant.

The processor 5 drives the motorizations 4 to facilitate heed of the trajectory taken by the instrument 2, when it is manipulated by the operator. Therefore the mechanical structure 3, under the action of the motorizations 4, applies a force allowing the manipulation by the operator to be urged and guided towards the desired trajectory.

When the target is reached by the instrument 2, but the operator continues to move the instrument 2, the processor 5 leads to application of a reactive force in the opposite direction, or warns the user by a vibration or sound signal.

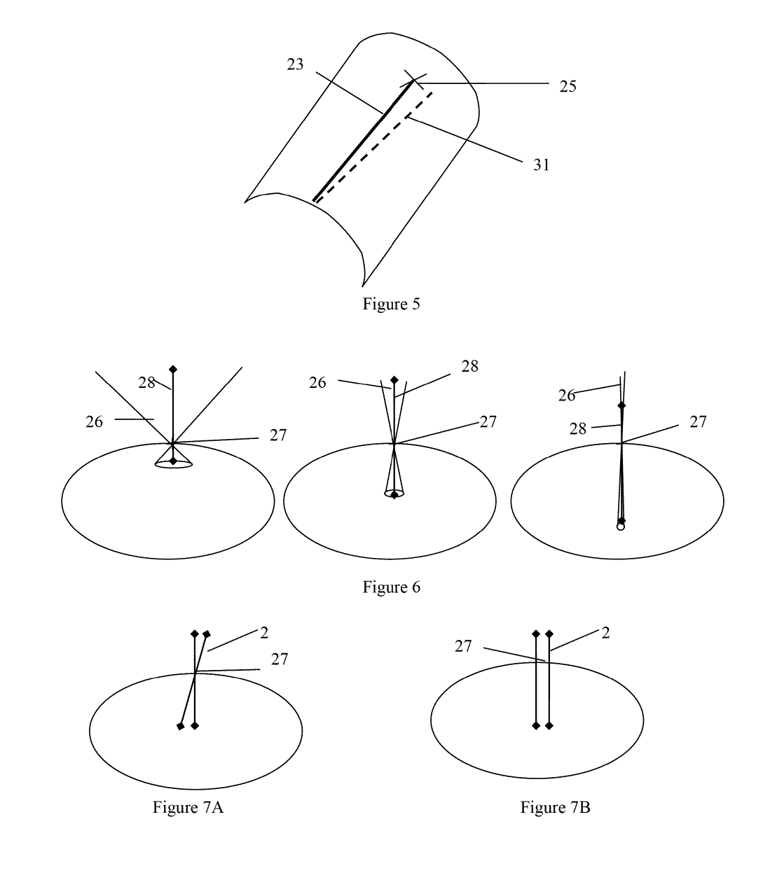

In an embodiment, the assistive assembly 6 allows the display (on screen 21 of the processing unit or on another screen of the assembly) and in accordance with an above-mentioned position-informing system 36, of the current trajectory 31 and of the desired trajectory 23 towards the target 25 (cf. FIG. 5). Visual effects (colours, alarms) can be used on the screen to indicate any deviation to the operator.

In some cases of emergency, it may be necessary for the operator to remove the instrument 2 rapidly from the patient's body.

For this purpose, the processor 5 drives the motorizations 4 so as, firstly, to facilitate heed of the kinematic constraint on the instrument and secondly, to enable the instrument not follow this constraint if the operator performs a given manipulation (such as manipulation using force exceeding a threshold, or velocity greater than a threshold). In this case, the instrument is allowed not to observe the kinematic constraint, thereby enabling the operator to withdraw the instrument 2 from the patient's body, in particular in the event of an emergency.

### 3^{rd} Example of Embodiment

As already mentioned, the guide within which the instrument is compelled to position itself may be defined dynamically. In particular, the guide may be defined dynamically in relation to the entry of the instrument 2 into an organ or patient region.

For example, for anaesthesia, it is possible to define a cone 26 which is reduced as and when the instrument 2 enters into the organ to be anesthetized (cf. FIG. 6). The region close to the skin is more sensitive, and a larger amount of anesthetizing substance is necessary whilst the deeper regions require a smaller amount.

It is also possible to exert a kinematic constraint on the instrument 28, forcing it to remain on a given point of insertion 27, irrespective of entry of the instrument 2 in the organ. Only rotation about a point of insertion is authorized with a greater or lesser apex angle depending on cases.

In an embodiment, the device 1 automatically changes over from the mode described in the 1st example (choice of point of insertion) to the mode described in the 3rd example (controlled insertion and holding at the point of insertion) when it is detected that the instrument 2 is close to the organ. Alternatively, the operator selects this mode manually via the interface 20 of the assembly 6.

In some cases, the instrument must be withdrawn during the procedure so that it can be reloaded. This is the case for example with a needle used for anaesthesia. Before withdrawing the instrument 2, the device 1 records the point where movement of the instrument 2 was stopped. After reinsertion of the instrument 2, the device 1 guides the instrument 2 towards this stop point to resume the procedure at the point where it was halted.

As it can be ascertained, the guiding of the instrument 2 is virtual, and is not based on a physical guide as in numerous cases in the prior art.

### Other Examples of Embodiment

In some cases, a three-dimensional map of patient regions to be avoided is drawn up on the basis of images taken by the medical imaging system 11. This map is produced by the processing unit 29 for example of the assistive assembly 6 which processes the images taken by the imaging system 11 and identifies the regions to be avoided.

The guide corresponds to a geometric region enabling the instrument to avoid the regions identified in the patient (vessels, etc.).

If there is a risk that the operator may touch a region that is to be avoided, the processor acts on the motorizations to make insertion more difficult (the resistance exhibited by the structure vis-à-vis the operator is controlled for example so that it depends on the penetration along the axis) or to deflect the insertion trajectory or to trigger a vibration.

In addition to the patient regions to be avoided, the guide is, in an embodiment, defined to enable the instrument to avoid physical objects, such as parts of the imaging system for example (compression paddle, detector, etc.).

In an embodiment, the device and/or the processing unit 29 of the assembly 6 are configured to receive a plurality of data items related to the position of physical objects and/or predetermined patient regions: position of the compression paddle, position of the detector, geometry of the imaging system, position of the X-ray tube, regions identified in the images taken by the imaging system, patient height, etc. This data is communicated by wire or wireless communication means.

In one embodiment, the assembly 6 comprises a visualization screen 21 allowing the display both of images taken by the imaging system 11 and of calculated data (real-time position of instruments, regions to be avoided, position of organs, recommended trajectories, etc.).

In one embodiment, it is sought to know exactly the size of the instrument, such as the length of the needle for example.

The operator moves the instrument towards a predetermined position, and sets the instrument in movement with various orientations. The device records the position and the various orientations of the instrument via a position sensor integrated in the device. The processor 5 or the processing unit 29, via a processing programme, can determine the dimensions of the instrument, such as its length, in particular by determining the intersection of the different directions of the instrument 2.

In one embodiment which may or may not be complementary with the previously described embodiments, the device 1 imposes a kinematic constraint on the instrument 2 to form a guide allowing the instrument to be maintained at the point of insertion, whilst authorizing rotational movements about this point of insertion. This avoids having to enlarge the insertion point 27 for the instrument (cf. FIG. 7A), during manipulations by the operator to draw the instrument close to the target. This therefore avoids additional discomfort for the patient. If translation of the instrument 2 was authorized, the point of insertion would be enlarged, which would be a source of discomfort for the patient (cf. FIG. 7B). In addition, the bleeding caused by insertion of the instrument into the patient is reduced.

Since the kinematic constraint allows the instrument to be maintained at the point of insertion whilst authorizing rotational movements about this point, this may be useful in numerous cases and enables the operator to correct the trajectory.

This may be useful for example if the target to be reached has moved. Knowledge of the movement of the target can be obtained by taking new images with the imaging system 11. Another, non-limiting, application concerns the case in which the operator wishes to change the trajectory for example to avoid obstacles.

In all cases, the guide formed by the kinematic constraint allows the instrument to be maintained in the point of insertion despite the movements imposed upon the instrument 2 by the operator.

The position of the point of insertion can be determined by means of images taken by the imaging system 11, allowing identification of the surface of the organ in which the instrument is inserted. Alternatively, the position of the point of insertion is visualized by means of an optical system e.g. of laser type. Alternatively, it is the operator who indicates to the assembly 6 the position of the point of insertion, via the interface 20.

In an embodiment, the device 1 is capable of receiving a plurality of instruments, to perform different phases of the procedure. The fixing of the instruments on the device is, in an embodiment, configured to allow changeover from one instrument to another during the procedure.

In some cases, the device comprises a system for identifying the instruments, for example by reading off a bar code, or RFID chip read-off. Alternatively, it is the operator who manually indicates to the device the type of on-board instruments.

Alternatively, it is a combination of data entered by the operator and of data read by the device.

One advantageous but non-limiting application concerns mammography procedures. In this case, the imaging system is a mammography unit, e.g. a tomosynthesis mammography unit. A mammary procedure notably comprises an anaesthesia step, a biopsy step and the placing of an instrument in the patient's body. The instrument is most often a needle.

One embodiment also concerns a computer program product, which can be loaded in the memory of the processing unit 29 of the assembly 6, or in the processor 5 of the device and capable of computing guides for the instrument 2 such as previously described. In general, these guides are computed from images taken by the imaging system, by analysis and processing of the images. As previously seen, these guides can be computed dynamically in relation to various parameters.

The embodiments which have been described in the present description can be implemented using the same assistive device and/or assistive assembly, or by separate devices/assemblies.

These embodiments can be implemented separately, or they can be combined together in full or in part in any technically possible combination.

The device 1 and the assembly 6 may be configured to implement one or more of these embodiments.

This written description uses examples to disclose the various embodiments, including the best mode, and also to enable any person skilled in the art to practice the embodiments, including making and using any devices or systems and performing any incorporated methods. The patentable scope of the embodiments is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for an assisted manipulation of an instrument in an assistive assembly for instrument manipulation, wherein the assistive assembly comprises a medical imaging system comprising an immobilizing support to support at least one part of a patient intended to be imaged or already imaged by the medical imaging system, an assistive device for the assisted manipulation of the instrument intended to be used in the patient, wherein the assistive device comprises a mobile mechanical structure operable by an operator on which at least one instrument can be fixed, the method comprising:
calibrating the position of the assistive device relative to the position of the medical imaging system, thereby allowing the position of the at least one part of the patient to be known within a reference frame related to the assistive device, to assist the operator when manipulating the instrument in the patient.

2. The method according to claim 1, further comprising providing the operator with information characterizing the position of the assistive device and/or of the instrument relative to the at least one part of the patient or to a target of the at least one part of the patient.

3. The method according to claim 1, wherein the assistive assembly further comprises:
motors configured to actuate the mobile mechanical structure over one or more degrees of freedom, and
a processor configured to drive the motors to facilitate a meeting of at least one kinematic constraint on the instrument, the meeting of the at least one kinematic constraint being achieved by cooperation between manipulations by the operator and actions by the motors in response to the manipulations, wherein the calibration enables the processor to transform the at least one kinematic constraint on the instrument defined in a reference frame of the medical imaging system, into the reference frame of the assistive device (1), so as to drive the motors using the transformed at least one kinematic constraint.

4. The method according to claim 1, wherein the assistive device and the medical imaging system are combined and the calibration is pre-programmed.

5. The method according to claim 1, wherein the calibration is determined by the operator, prior to performing a procedure on the patient, and comprises:
positioning the assistive device at points of the medical imaging system, the position of the points in the reference frame of the medical imaging system being transmitted to a calibration system calibrating the position of the assistive device relative to the position of the medical imaging system, and
inferring therefrom the calibrated position of the assistive device relative to the medical imaging system, via processing by the calibration system.

6. The method according to claim 3, wherein the processor is further configured to drive the motors so as to compel the instrument to position the instrument within a guide, corresponding to a geometric region in space.

7. The method according to claim 6, wherein the guide is defined dynamically, in relation to:
kinematic parameters of the instrument, and/or
manipulations of the instrument performed by the operator.

8. The method according to claim 6, wherein the guide is defined dynamically in relation to a deformation model of a region of the patient in which the instrument has been inserted.

9. The method according to claim 6, wherein the guide is re-computed in relation to information derived from images of the patient, taken by the medical imaging system.

10. The method according to claim 6, wherein the guide corresponds to a geometric region allowing the instrument, when the instrument is being moved by the operator, to avoid physical objects or predetermined regions of the patient.

11. The method according to claim 3, wherein the at least one kinematic constraint on the instrument is defined by the operator using images provided by the medical imaging system, via an interface of the assistive assembly.

12. An assistive assembly for instrument manipulation, the assistive assembly comprising:
a medical imaging system comprising:
a support to immobilize at least one part of a patient intended to be imaged or already imaged by the medical imaging system; and
an assistive device for the manipulation of an instrument intended to be used in the patient, the assistive device comprising a mobile mechanical structure operable by an operator, on which at least one instrument can be fixed; and
at least one calibration system configured to calibrate the position of the assistive device relative to the position of the medical imaging system, thereby allowing the position of the at least one part of the patient to be known within a reference frame related to the assistive device to assist the operator in the manipulation of the instrument in the patient.

13. The assembly according to claim 12, further comprising:
motors actuating the mobile mechanical structure over one or more degrees of freedom; and
a processor configured to drive the motors to facilitate the meeting of at least one kinematic constraint on the instrument, the meeting of the at least one kinematic constraint being achieved by cooperation between manipulations by the operator and actions by the motors in response to the manipulations,
wherein the at least one calibration system is further configured to enable the processor to transform the at least one kinematic constraint on the instrument defined in a reference frame of the medical imaging system into the reference frame of the assistive device, so as to drive the motors using the transformed at least one kinematic constraint.

14. (canceled)
